(19) United States
(12) Patent Application Publication
(10) Pub. No.: US 2003/0217418 A1
(43) Pub. Date: Nov. 27, 2003

(54) Hair dye composition and method for producing same

(76) Inventors: Addi Fadel, Hoboken, NJ (US); Parbhubhai Patel, Belleville, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/156,207

(22) Filed: May 24, 2002

(51) Int. Cl. A61K 7/13

(52) U.S. Cl. 8/405; 8/406; 8/412; 8/421

(57) ABSTRACT

The present invention is directed to a composition for a new 3-nitro 4-aminophenol of the general formula:

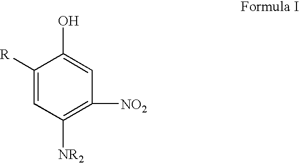

wherein R denotes a hydroxy-alkyl group. The instant invention also pertains to a process of manufacturing new dyes, for coloring keratinic fibers, that comprise at least one compound of Formula I above.

### BACKGROUND OF THE INVENTION

The present invention is directed to a composition for a new 3-nitro 4-aminophenol of the general formula:

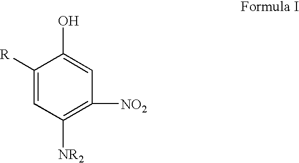

wherein R denotes a hydroxy-alkyl group. The instant invention also pertains to a process of manufacturing new dyes, for coloring keratinic fibers, that comprise at least one compound of the Formula I above.

### DISCUSSION OF RELATED ART

Hair coloration by both men and women comprises a significant portion of the hair care business in the United States. Coloring agents containing oxidation dyes are well known because oxidation dyes are suitable for yielding specific color shades and because they cover the desired areas very well.

In addition to oxidation dyes, coloring agents belonging to the family of nitroaminophenols have received wide acceptance within the hair care industry. For example, U.S. Pat. No. 4,740,622 is directed to 3-amino 4-nitrophenols, the process for its preparation and its use as a coloring agent for dyeing human hair.

U.S. Pat. Nos. 4,125,601 and 4,575,378 are directed to dyes comprising 4-(α-hydroxypropyl)-amino 3-nitrophenol, 4-(β-hydroxyethyl)-amino 3-nitrophenol and 4-(β-hydroxypropryl)-3-nitrophenol. In each of these dyes, there exists only a single substituted group on the para-amino position of the corresponding 4-amino 3-nitrophenol.

There are several advantages of dyes that contain the compound of the general structure of Formula (I) above. First, the process of manufacturing the dyes of the instant invention requires only a single step process, in contrast to the two-step processes known in the art.

Another advantage is that the epoxidation of the instant invention is more economical than is the processes presently used to synthesize 4(α-hydroxypropyl)-amino or 3-nitrophenol 4-(β-hydroxyethyl)amino 3-nitrophenol. Better performance in general and increases in non-ionic interactions with hair surfaces allows increased wash fastness. Also, better stability may also be experienced. Accordingly, there is a need for synthesizing aminophenol dyes using a single step process.

### SUMMARY OF THE INVENTION

Epoxides are used in many other processes for the synthesis of semi-permanent dyes because they are an important source of electrophilic carbon, a carbon that is highly susceptible to attack by a wide variety of nucleophiles. The manufacturing process according to the invention is characterized in that an epoxide is reacted with 4-amino 3-nitrophenol in an aqueous environment at a high temperature and in some conditions at high pressure. An epoxide undergoes a nucleophillic attack by an amino group at the para position of 4-amino 3-notriphenol resulting in a base-catalyzed cleavage of the epoxide.

### DETAILED DESCRIPTION OF THE INVENTION

In a first preferred composition, 4-Bis N₁, N₂(2-hydroxyethylamino) 3-Nitrophenol:

Dissolve 80 grams (0.52 mol) of 3-nitro 4-aminophenol in 400 ml water. Then, transfer the mixture to a pressure-sealed autoclave, and add 10 grams sodium mono-phosphate. Heat the mixture to approximately 75-80° C. In small increments, add 24 grams of ethylene oxide in one-hour intervals at 20 psi pressure. Check for reaction completion after about 3 hours using thin layer chromatography. If unreacted material is still present, maintain conditions and check again about every 30 minutes or thereabout, until the reaction is completed. Unload the reaction mass and then cool to about 10° C.-15° C. Thereafter, allow the material to crystallize and wash the resulting precipitate twice with 25 ml water. Dry under vacuum at 60° C.-80° C. Melting point of product approximately: 140° C.-142.° C.; weight of product approached 48 grams.

The general reaction may be represented as follows:

4-Bis N₁, N₂-(2-hydroxyethylamino) 3-Nitrophenol

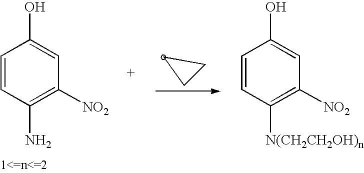

In a second preferred composition, 4-Bis N₁, N₂-(β-hydroxypropyl) 3-Nitrophenol:

Dissolve 20 grams of 4-amino 3-nitrophenol (0.13 moles) and 72 grams of propylene oxide (1.24 moles) in water in a flask equipped with a reflux condenser. Heat the mixture to about 65° C.-70° C. for about 4 hours and check reaction completion using thin layer chromatography. If unreacted material is still present, maintain the heat conditions and check for reaction completion every 30 minutes or thereabout. Cool the mixture by cooling to about 10° C.-15° C. and allow the crystals to precipitate. Filter the material and dry it using a vacuum. Reprecipitate the material using ethyl acetate. Thereafter, dry the precipitate in an oven at about 60° C.-70° C.

Melting Point of Product: 126-128° C. Weight of Product: 17.4 g

In general, the reaction may be represented as follows:

4-Bis N₁, N₂-(β-hydroxypropyl) 3-Nitrophenyl

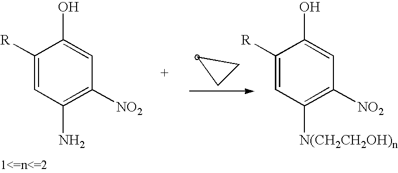

While the foregoing description and compounds represent the preferred embodiments of the present invention, it will be understood that various changes and modifications may be made without departing from the spirit and scope of the present invention, as recited in the following claims.

What is claimed is:

1. A hair coloring dye comprising 4-Bis N₁, N₂-(2-hydroxyethylamino) 3-nitrophenol.

2. A hair coloring dye comprising 4-Bis N₁, N₂-(β-hydroxypropyl) 3-nitrophenol.

3. A method for producing 4-Bis N₁, N₂-(2-hydroxyethylamino) 3-nitrophenol comprising the steps of:
a. providing a solution of 4-amino 3-nitrophenol;
b. adding sodium mono-phosphate to the solution of step (a);
c. adding ethylene oxide to the solution of step (b);
d. allowing the composition of step (c) to precipitate.

4. The method of claim 3 further comprising the stop of washing the precipitate with water.

5. A method for producing 4-Bis N₁, N₂ (B-hydroxypropyl) 3-Nitrophenol comprising the steps of:
a. dissolving about 20 g of 4-amino 3-nitrophenol and about 72 grams of propylene oxide in water to form a solution;
b. heating the solution of step (a) to a temperature between about 65° C. and about 70° C.;
c. cooling the mixture of step (b) to a temperature between about 10° C. and 15° C. to allow precipitation;
d. filtering the precipitate of step (c);
e. drying the precipitate of step (d);
f. reprecipitating the material of step (e) with ethylacetate; and
g. drying the material of step (f).

6. The method of claim 5, wherein step (b) is carried out while maintaining a pressure of about 20 psi.

7. A method for producing 4-Bis N₁, N₂-(2- hydroxyethylamino) 3-nitrophenol comprising the steps of:
a. dissolving about 80 grams of 4-amino 3-nitrophenol in about 400 ml water to yield a solution;
b. adding about 10 grams of sodium mono-phosphate to the solution of step (b);
c. heating the solution of step (b);
d. adding about 24 grams of ethyl oxide in small increments into the solution of step (c) in approximate one hour intervals while maintaining a pressure of the solution at about 20 psi;
e. maintaining the pressure of the mixture of step (d) at temperature conditions until substantially all of the mixture is reacted;
f. cooling the reaction mixture of step (e);
g. allowing the material of step (f) to form a precipitate.

8. The method of claim 7, further comprising washing the precipitate with water.
